(19) United States
(12) Patent Application Publication
(10) Pub. No.: US 2026/0272768 A1
(43) Pub. Date: Sep. 17, 2026

(54) INTEGRATED SYSTEM AND METHOD FOR GESTURE-BASED TIPPING

(71) Applicant: HYTTO PTE. LTD, Singapore (SG)

(72) Inventors: Dan LIU, Singapore (SG); Jilin QIU, Singapore (SG); Jiajun LUO, Guangzhou (CN); Shiren YANG, Guangzhou (CN)

(73) Assignee: HYTTO PTE. LTD, Singapore (SG)

(21) Appl. No.: 19/682,583

(22) Filed: May 20, 2026

Related U.S. Application Data
(63) Continuation-in-part of application No. 19/320,005, filed on Sep. 5, 2025.
Application No. 19/320,005 is a continuation-in-part of application No. 18/494,128, filed on Oct. 25, 2023, now abandoned.
(63) Continuation-in-part of application No. 19/350,878, filed on Oct. 6, 2025.
Application No. 19/350,878 is a continuation-in-part of application No. 18/928,125, filed on Oct. 27, 2024, now Pat. No. 12,433,816.
Application No. 19/350,878 is a continuation-in-part of application No. 18/825,346, filed on Sep. 5, 2024, now Pat. No. 12,667,513.
(63) Continuation-in-part of application No. 17/934,290, filed on Sep. 22, 2022.
Application No. 17/934,290 is a continuation-in-part of application No. 17/579,839, filed on Jan. 20, 2022, now Pat. No. 12,171,708.
Application No. 17/579,839 is a continuation-in-part of application No. 17/717,917, filed on Apr. 11, 2022, now Pat. No. 11,938,078.
Application No. 17/717,917 is a continuation of application No. 16/352,876, filed on Mar. 14, 2019, now Pat. No. 11,311,453.
Application No. 17/934,290 is a continuation-in-part of application No. 16/835,808, filed on Mar. 31, 2020, now Pat. No. 11,452,669.
(60) Provisional application No. 62/830,195, filed on Apr. 5, 2019.
(60) Provisional application No. 63/594,930, filed on Oct. 31, 2023.
(60) Provisional application No. 63/594,930, filed on Oct. 31, 2023.

(51) Int. Cl. A61H 19/00; A61F 5/41; A61H 23/02

(52) U.S. Cl. CPC A61H 19/44; A61F 5/41; A61H 19/34; A61F 2005/417; A61H 23/02; A61H 2201/5007; A61H 2201/5071; A61H 2201/5097

(57) ABSTRACT

A system and method for interactive tipping in a live streaming environment are disclosed. A viewer device displays, on a first interface, a representation of a tipping input, and displays, on a second interface, a live streaming session. During access to the live streaming session, a pointing operation is detected with respect to the representation of the tipping input, wherein the pointing operation comprises a gesture originating from a starting position within the first interface and directed toward outside the first interface. In response to the pointing operation, a tipping input associated with a host user of the live streaming session is activated. The system further generates visual effects rendered on the second interface based on the pointing operation, and maps kinematic parameters of the pointing operation to control instructions for an interactive device associated with the host user, thereby producing a physical response corresponding to the gesture.

### CROSS-REFERENCE TO RELATED APPLICATIONS

This application is a Continuation-in-Part of U.S. application Ser. No. 19/320,005, filed Sep. 5, 2025, which is a Continuation-in-Part of U.S. application Ser. No. 18/494,128, filed Oct. 25, 2023, now abandoned. This application is also a Continuation-in-Part of U.S. application Ser. No. 19/350,878, filed Oct. 6, 2025, which is a Continuation-in-Part of U.S. application Ser. No. 18/928,125, filed Oct. 27, 2024, now U.S. Pat. No. 12,433,816, issued Oct. 7, 2025, which claims the benefit of Provisional Application No. 63/594,930, filed Oct. 31, 2023. U.S. application Ser. No. 19/350,878 is also a Continuation-in-Part of U.S. application Ser. No. 18/825,346, filed Sep. 5, 2024, which claims the benefit of Provisional Application No. 63/594,930, filed Oct. 31, 2023. This application is also a Continuation-in-Part of U.S. application Ser. No. 17/934,290, filed Sep. 22, 2022, which is a Continuation-in-Part of U.S. application Ser. No. 17/579,839, filed Jan. 20, 2022, now U.S. Pat. No. 12,171,708, issued Dec. 24, 2024. U.S. application Ser. No. 17/934,290 is also a Continuation-in-Part of U.S. application Ser. No. 17/717,917, filed Apr. 11, 2022, now U.S. Pat. No. 11,938,078, issued Mar. 26, 2024, which is a Continuation of U.S. application Ser. No. 16/352,876, filed Mar. 14, 2019, now U.S. Pat. No. 11,311,453, issued on Apr. 26, 2022. U.S. application Ser. No. 17/934,290 is also a Continuation-in-Part of U.S. application Ser. No. 16/835,808, filed Mar. 31, 2020, now U.S. Pat. No. 11,452,669, issued Sep. 27, 2022, which claims the benefit of Provisional Application No. 62/830,195, filed Apr. 5, 2019. The entire disclosure of all of these applications is incorporated herein by reference.

### FIELD OF THE INVENTION

The present invention relates generally to interactive live streaming systems, and more particularly, to systems and methods for enabling gesture-based virtual tipping through natural body-motion interactions on a viewer device, with synchronized visual effects rendered on a streaming display and haptic feedback transmitted to a performer-side interactive toy.

### BACKGROUND OF THE INVENTION

Live streaming platforms have become a significant medium for real-time interaction between content creators and audiences. Viewers typically engage with performers through text-based chat, virtual gift mechanisms, and other digital interaction modalities. Tipping, in particular, has emerged as a primary revenue model for many live streaming ecosystems, wherein viewers send virtual currency or digital gifts to performers during a live broadcast session.

In conventional live streaming tipping implementations, a viewer initiates a tip by selecting a predefined virtual gift icon from a menu and confirming a transaction through one or more button taps. The tipping interaction is generally limited to a discrete, transactional click-to-send paradigm. The visual feedback associated with such conventional tipping is typically a static or pre-animated graphic overlay that bears no dynamic relationship to the viewer's physical input. Furthermore, where performer-side connected devices are employed, the actuation parameters of such devices are conventionally determined solely by the monetary value of the tip, without regard to the kinematic characteristics of the viewer's input gesture. At present, there is limited integration between the physical expressiveness of a viewer's tipping action, the real-time visual representation of that action, and the tactile response experienced on the performer side.

Accordingly, it would be desirable to provide a system and method that enables a more immersive, physically expressive, and bidirectionally interactive tipping experience in a live streaming environment.

The exemplary disclosed system and method are directed to overcoming one or more of the shortcomings set forth above and/or other deficiencies in existing technology.

### SUMMARY OF THE INVENTION

The present invention provides an integrated system and method for gesture-based tipping in a live streaming environment, wherein a viewer performs natural body-motion operations on a viewer device to initiate and control virtual tipping, with real-time dynamic visual feedback rendered on both the viewer device and a streaming display, and with optional haptic linkage to a performer-side interactive toy driven by kinematic parameters of the viewer's gesture rather than solely by tip monetary value. The present invention enhances immersion, emotional connection between viewers and performers, and interactive engagement without requiring specialized hardware beyond commonly available consumer devices.

### BRIEF DESCRIPTION OF THE DRAWINGS

FIG. 1 is a system architecture diagram illustrating the overall components of the integrated gesture-based tipping system;

FIG. 2 is a schematic diagram depicting a first exemplary deployment configuration in which the viewer-side device comprises a separate display device presenting a second interface and a separate handheld device presenting a first interface;

FIG. 3 is a schematic diagram depicting a second exemplary deployment configuration in which a single touchscreen device concurrently presents the first interface and the second interface in distinct regions of the same display;

FIG. 4 is a detailed illustration of a swipe-to-scatter tipping mode;

FIG. 5 is a detailed illustration of gesture-based tipping gesture performed on the first interface and visual effect rendering modes on the second interface;

FIG. 6 is a detailed illustration of a tilt-to-pour tipping mode, showing the first interface with a virtual vessel graphic and gyroscope-driven liquid-level animation;

FIG. 7 is diagram illustrating multiple visual effect rendering modes on the second interface, including viewer-local rendering and broadcaster-side rendering visible to all viewers;

FIG. 8 is a flow diagram illustrating an exemplary method for interactive tipping based on a pointing operation performed with respect to a representation of a tipping input on a first interface.

### DETAILED DESCRIPTION AND INDUSTRIAL APPLICABILITY

The following detailed description sets forth various embodiments of the present invention. It is to be understood that the specific embodiments described herein are shown by way of illustration and not as limitations of the invention. The principal features of this invention can be employed in various embodiments without departing from the scope of the invention. All parts and percentages are by weight unless otherwise specified. In some embodiments, the described functionality may be implemented in software, hardware, or a combination thereof, and may be distributed across one or more devices or system components. The specific allocation of functionality is not limited to the exemplary configurations described herein.

As used herein, the term “processor” refers to any general-purpose or special-purpose computing unit capable of executing instructions, including but not limited to a central processing unit, a microcontroller, a digital signal processor, a graphics processing unit, a system-on-chip, or a field-programmable gate array. The term “storage” or “memory” refers to any non-transitory computer-readable medium capable of storing data and instructions, including but not limited to random access memory, flash memory, solid-state drives, hard disk drives, optical media, or any combination thereof. The term “communication unit” refers to any hardware or software component capable of transmitting and receiving data over a network, including but not limited to a wireless transceiver, a network interface, a Bluetooth module, a Wi-Fi adapter, a cellular modem, or any combination thereof. The term “sensor” refers to any hardware component capable of detecting physical phenomena and converting them into electrical signals, including but not limited to a touchscreen digitizer, a gyroscope, an accelerometer, a magnetometer, a barometer, a proximity sensor, or any combination thereof. The term “representation of a tipping input” or “image of a tipping input” or “tipping input image” as used in the context of the first interface refers to any visual element rendered on a display, including but not limited to a graphical icon, an animated sprite, a bitmap, a vector graphic, a photograph, or a stylized representation of a physical object such as currency, a coin, a token, a beverage container, or any other thematic graphic. The term “pointing operation” refers to any user input gesture that has a discernible spatial vector component, including but not limited to a swipe, a drag, a flick, a fling, or a tilt. In some embodiments, the pointing operation is not limited to continuous motion gestures, and may further include discrete or compound input actions that indicate a directional intent relative to the first interface, including combinations of touch, motion, or sensor-based inputs.

### 1. System Architecture Overview

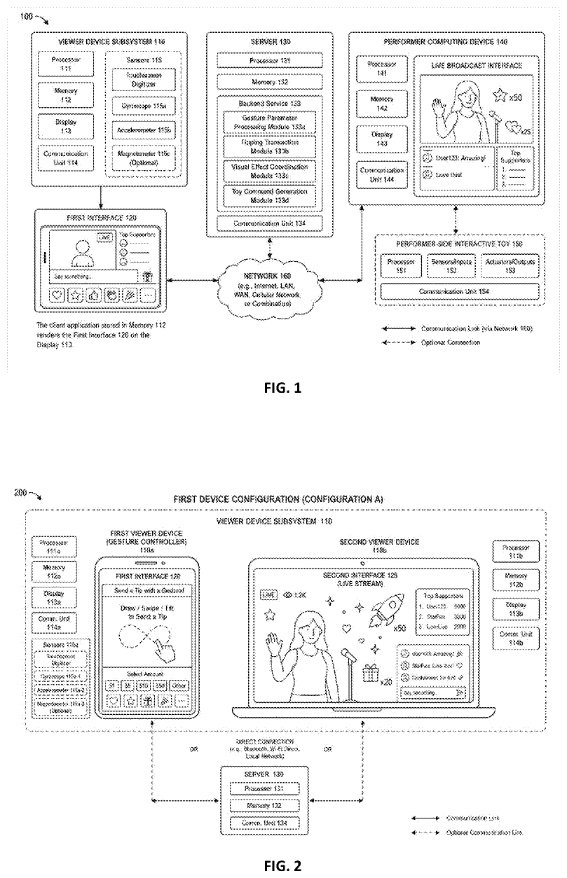

Referring now to FIG. 1, the integrated gesture-based tipping system 100 comprises a viewer device subsystem 110, a server 130, a performer computing device 140, and optionally a performer-side interactive toy 150. The viewer device subsystem 110 includes at least one viewer device having a processor 111, a memory 112, a display 113, a communication unit 114, and one or more sensors 115. The sensors 115 include at least a touchscreen digitizer associated with the display 113. In some embodiments, the sensors 115 further include a gyroscope 115a, an accelerometer 115b, and optionally a magnetometer 115c. The viewer device subsystem 110 is configured to execute a client application stored in the memory 112, the client application rendering a first interface 120 on at least a portion of the display 113. The server 130 includes a processor 131, a memory 132, and a communication unit 134. The server 130 is communicatively coupled to the viewer device subsystem 110 and the performer computing device 140 via a network 160. The network 160 may comprise the Internet, a local area network, a wide area network, a cellular network, or any combination thereof. The server 130 is configured to execute a backend service stored in the memory 132, the backend service including a gesture parameter processing module 133a, a tipping transaction module 133b, a visual effect coordination module 133c, and a toy command generation module 133d. The performer computing device 140 includes a processor 141, a memory 142, a display 143, and a communication unit 144. The performer computing device 140 is configured to execute live streaming software that renders a live broadcast interface on the display 143. The performer computing device 140 receives visual effect instructions from the server 130 and renders tipping visual effects on the live broadcast interface. The performer-side interactive toy 150 includes a processor 151, a memory 152, an actuator 153 (such as a vibration motor, a linear resonant actuator, an eccentric rotating mass motor, or a piezoelectric actuator), and a communication unit 154. In a preferred embodiment, the communication unit 154 comprises a Bluetooth Low Energy (BLE) transceiver that communicatively couples the toy 150 to the performer computing device 140. In alternative embodiments, the communication unit 154 may comprise a Wi-Fi transceiver, a Zigbee transceiver, a USB wired connection, or any other short-range or long-range communication interface. The toy 150 receives actuation commands from the server 130, either directly via the network 160 or indirectly through the performer computing device 140, and drives the actuator 153 in accordance with the received commands.

### 2. Device Configurations

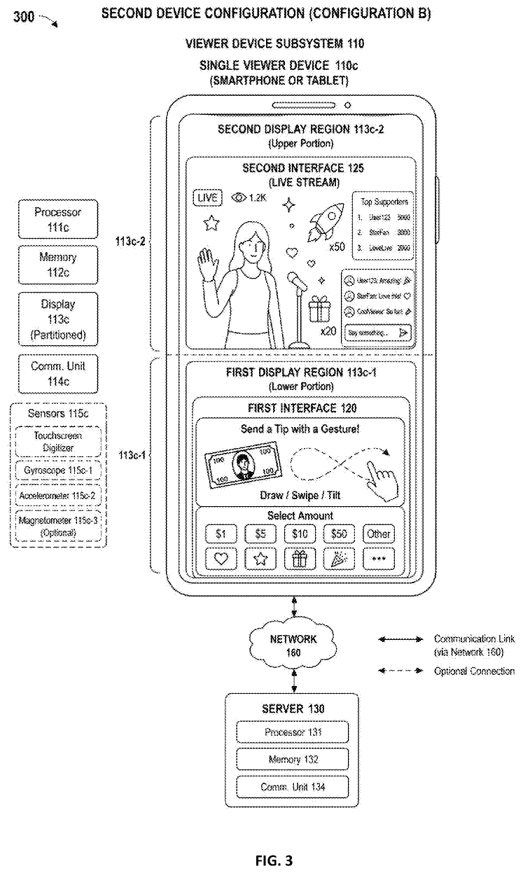

Referring now to FIG. 2, in a first device configuration (Configuration A), the viewer device subsystem 110 comprises a first viewer device 110a and a second viewer device 110b. The first viewer device 110a is a handheld device such as a smartphone or a tablet, and serves as a gesture controller. The first viewer device 110a renders the first interface 120 on its display 113a. The second viewer device 110b is a computing device such as a desktop computer, a laptop computer, a smart television, or a tablet, and renders a second interface 125 on its display 113b. The second interface 125 displays the live stream broadcast from the performer (also referred to herein as host user). The first viewer device 110a and the second viewer device 110b are communicatively coupled to each other, either directly (e.g., via Bluetooth, Wi-Fi Direct, local network) or indirectly through the server 130, such that gesture events detected on the first viewer device 110a trigger visual effects on the second interface 125 rendered on the second viewer device 110b.

In one aspect, the connection between the first viewer device 110a and the second viewer device 110b is established by the viewer initiating a pairing procedure within the client application. In some embodiments, the pairing procedure comprises scanning a QR code displayed on the second viewer device 110b using a camera of the first viewer device 110a. In other embodiments, the pairing procedure comprises entering a numeric code, tapping a near-field communication (NFC) tag, or authenticating through a shared user account on the server 130.

Referring now to FIG. 3, in a second device configuration (Configuration B), the viewer device subsystem 110 comprises a single viewer device 110c, such as a smartphone or a tablet. The display 113c of the single viewer device 110c is partitioned into a first display region 113c-1 and a second display region 113c-2. The first interface 120 is rendered in the first display region 113c-1, and the second interface 125 is rendered in the second display region 113c-2. In a preferred embodiment, the first display region 113c-1 occupies a lower portion of the display 113c and the second display region 113c-2 occupies an upper portion of the display 113c. In alternative embodiments, the spatial arrangement may be reversed, side-by-side, or user-configurable. In some embodiments, the first interface 120 is rendered as an overlay layer on top of the second interface 125, with adjustable transparency. In some embodiments, the first display region 113c-1 and the second display region 113c-2 are resizable by the viewer through a drag handle positioned at the boundary between the two regions.

In a third device configuration (Configuration C), the viewer device subsystem 110 comprises a single viewer device 110c, and the first interface 120 is rendered as a modal or semi-modal panel that the viewer can invoke and dismiss by a predefined gesture (such as a long press, a multi-finger tap, or a swipe from an edge of the display). When the first interface 120 is invoked, it overlays a portion of the second interface 125. When dismissed, the second interface 125 occupies the full display.

In some embodiments, the second interface is spatially separate from, and located outside the first interface. The pointing operation is interpreted as being directed toward outside the first interface based on a directional trend of the input, even if the input does not physically extend beyond a boundary of the first interface. For example, a gesture approaching or targeting a boundary region of the first interface may be classified as being directed toward outside the first interface. In some embodiments, the live streaming session presented on the second interface includes content associated with a host user, such that the host user is visually or functionally represented within the second interface.

### 3. First Interface and Image Element

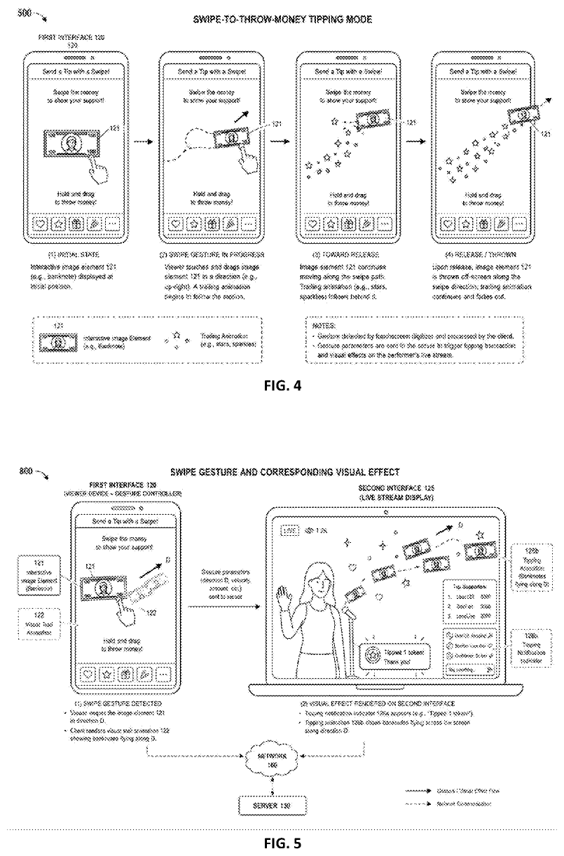

Referring now to FIG. 4, the first interface 120 rendered on the viewer device includes at least one interactive image element 121. The image element 121 is a graphical representation that the viewer can manipulate through touch and motion gestures. The image element 121 constitutes a representation of a tipping input. In a preferred embodiment, the image element 121 is a representation of a banknote. In alternative embodiments, the image element 121 may be a representation of a coin, a gem, a token, a playing card, a flower, a heart, a star, a beverage container, or any other thematic graphic. In some embodiments, the viewer is presented with a selection interface allowing the viewer to choose among a plurality of image element themes. In a further embodiment, the plurality of image element themes includes banknote representations from different countries or denominations, such that the viewer may select a United States dollar bill, a Euro banknote, a Japanese yen note, a Chinese yuan note, a British pound note, or any other currency representation. The representation of the tipping input serves as an interactive element through which the viewer initiates or controls tipping actions via the pointing operation. The image element 121 is rendered at an initial position within the first interface 120. The image element 121 is responsive to touch input events detected by the touchscreen digitizer of the viewer device. In some embodiments, the image element 121 is further responsive to motion input events detected by the gyroscope 115a and the accelerometer 115b of the viewer device.

### 4. Gesture Detection and Parameter Extraction

The client application executing on the viewer device includes a gesture detection module that monitors sensor data from the sensors 115 and identifies predefined gesture types. For each detected gesture, the gesture detection module extracts a set of kinematic parameters (also referred to herein as physical parameters). The kinematic parameters include, but are not limited to: a gesture type identifier (e.g., swipe, tilt, tap, flick), a direction vector (e.g., an angle in degrees relative to a reference axis of the display), a velocity magnitude (e.g., in pixels per second or in meters per second squared for accelerometer-based gestures), a duration (e.g., in milliseconds), a pressure value (where supported by the touchscreen digitizer), a frequency (e.g., number of repeated gestures per unit time), and a positional coordinate (e.g., the starting point and ending point of a swipe gesture on the display coordinate system). In some embodiments, the input associated with the representation of the tipping input may be derived from non-gesture inputs, including automated inputs, preconfigured interaction patterns, or inputs generated by external systems, provided that such inputs are mapped to a directional intent relative to the first interface.

In one aspect, the gesture detection module applies a noise filter to raw sensor data before parameter extraction. In some embodiments, the noise filter comprises a low-pass Butterworth filter with a configurable cutoff frequency. In other embodiments, the noise filter comprises a Kalman filter or a moving average filter. The filtered sensor data is then processed by a gesture classifier. In a preferred embodiment, the gesture classifier is a rule-based state machine that transitions between idle, gesture-in-progress, and gesture-completed states based on threshold comparisons of sensor values. In an alternative embodiment, the gesture classifier is a trained machine learning model, such as a recurrent neural network or a convolutional neural network, that receives a time-windowed sequence of sensor readings and outputs a gesture classification and associated confidence score.

### 5. Tipping Mode I: Swipe-to-Scatter Mode

Referring now to FIG. 4, the swipe-to-scatter tipping mode is described in detail. This mode is the primary embodiment of the gesture-based tipping interaction.

### 5.1 Gesture Input

In the swipe-to-scatter mode, the viewer places a finger on the image element 121 displayed on the first interface 120. The image element 121 is positioned at an initial position P0 within the first interface 120. The viewer performs a directional swipe gesture, moving the finger from the initial position P0 along a trajectory toward a direction that extends beyond the boundary of the image element 121 or beyond the boundary of the first interface 120. The gesture detection module detects the swipe gesture and extracts the following kinematic parameters: a swipe direction vector D (defined as the angle θ between the swipe trajectory and a vertical reference axis of the display, measured in degrees), a swipe velocity V (defined as the displacement of the finger per unit time, measured in pixels per second), a swipe start coordinate (x0, y0), and a swipe end coordinate (x1,y1). In some embodiments, the swipe gesture does not necessarily extend beyond the boundary of the first interface. Instead, the swipe gesture may terminate at a location within the first interface while being directed toward a boundary region of the first interface. In such embodiments, the gesture detection module determines a directional intent of the swipe gesture based on a trajectory of the gesture, and classifies the swipe gesture as being directed toward outside the first interface even though an endpoint of the gesture remains within the first interface. For example, a swipe gesture that originates from the initial position P0 and proceeds toward an edge or corner region of the first interface, without crossing the boundary, may be interpreted as a valid pointing operation directed toward outside the first interface. In some embodiments, a predefined boundary proximity threshold is used, such that when an endpoint of the swipe gesture falls within a threshold distance from the boundary, the gesture is treated as being directed toward outside the first interface. In this manner, both (i) gestures that extend beyond the boundary of the first interface and (ii) gestures that remain within the first interface but are directed toward a boundary region may be recognized as pointing operations that satisfy directional criteria associated with cross-interface interaction. In some embodiments, the determination of whether the swipe gesture is directed toward outside the first interface is based on one or more of a direction vector, a velocity profile, and a positional relationship between the gesture endpoint and a boundary of the first interface.

In some embodiments, the gesture detection module further extracts a swipe pressure value (where the touchscreen supports pressure sensitivity or 3D Touch), a swipe contact area (the area of the finger contact patch on the touchscreen), and a swipe acceleration (the rate of change of swipe velocity over the duration of the swipe).

In a preferred embodiment, a single swipe gesture that crosses a predefined displacement threshold constitutes one tipping event. In some embodiments, the predefined displacement threshold is configurable by the platform operator and is stored as a parameter on the server 130. In other embodiments, the predefined displacement threshold is a fixed value, such as 50 pixels, 100 pixels, or 200 pixels.

In some embodiments, the viewer may perform a sustained press-and-hold gesture on the image element 121 without releasing the finger. While the press-and-hold gesture is maintained, the system continuously generates tipping events at a predefined rate (e.g., one tipping event per 500 milliseconds, one tipping event per second, or a rate that increases over the duration of the hold). The press-and-hold gesture thus enables a continuous tipping stream without requiring repeated swipe motions.

In some embodiments, the viewer may perform rapid successive swipe gestures on the image element 121. The gesture detection module detects each successive swipe as an independent tipping event. The frequency of tipping events corresponds to the frequency of the viewer's swipe gestures. A higher swipe frequency results in a higher tipping rate.

### 5.2 First Interface Visual Feedback

Upon detection of a swipe gesture on the image element 121, the client application renders a visual trail animation 122 on the first interface 120 FIG. 4. The visual trail animation 122 depicts the image element 121 (e.g., a banknote graphic) appearing to fly outward from the viewer's fingertip along the swipe direction vector D. In a preferred embodiment, the visual trail animation 122 includes a motion blur effect, a particle trail, and a gradual fade-out as the image element 121 moves toward the boundary of the first interface 120. In some embodiments, the visual trail animation 122 further includes a rotation of the image element 121 about its center axis, simulating the tumbling motion of a physical banknote in flight. The angular velocity of the rotation is proportional to the swipe velocity V.

In some embodiments, the visual trail animation 122 includes a plurality of smaller image elements (e.g., a fan of banknotes) that spread outward from the swipe origin, with the spread angle proportional to the swipe velocity V. In other embodiments, the visual trail animation 122 includes a sparkle or glitter particle effect overlaid on the trajectory of the image element 121.

After the visual trail animation 122 completes, the image element 121 is reset to the initial position P0 within the first interface 120, ready for the next gesture input.

### 5.3 Second Interface Visual Feedback

Referring now to FIG. 5, simultaneously with or shortly after the first interface visual feedback, the system renders a tipping visual effect 126 on the second interface 125. The tipping visual effect 126 includes at least: (a) a tipping notification indicator 126a that informs the viewer that a tipping transaction has been completed (e.g., a text overlay such as “Tipped 1 token” or a badge animation), and (b) a tipping animation 126b that depicts one or more image elements (e.g., banknotes) flying across the second interface 125 in a direction that corresponds to the swipe direction vector D detected on the first interface 120.

In one aspect, the correspondence between the swipe direction vector D and the direction of the tipping animation 126b on the second interface 125 is implemented as follows: the swipe direction angle θ detected on the first interface 120 is mapped to an entry direction on the second interface 125. For example, if the viewer swipes upward and to the left (θ≈315 degrees from the positive x-axis), the tipping animation 126b depicts banknotes entering the second interface 125 from the lower-right region and flying toward the upper-left region, or toward a performer region rendered on the second interface 125. In some embodiments, the mapping is a direct angular correspondence. In other embodiments, the mapping is a configurable transformation that accounts for the relative spatial arrangement of the first interface 120 and the second interface 125.

In a preferred embodiment, the velocity of the tipping animation 126b on the second interface 125 is proportional to the swipe velocity V detected on the first interface 120. A faster swipe results in a faster-moving tipping animation. In some embodiments, the number of image elements depicted in the tipping animation 126b is proportional to the swipe velocity V or to the swipe pressure value. A more forceful or faster swipe results in a greater number of banknotes depicted in the animation.

In some embodiments, the tipping animation 126b further includes an audio effect synchronized with the visual animation, such as a rustling paper sound, a cash register sound, a whooshing sound, or a chime.

### 5.4 Visual Effect Rendering Modes

Referring to FIG. 7, the system supports at least two visual effect rendering modes for the tipping animation 126b on the second interface 125.
Mode 1 (Viewer-Local Rendering): The tipping animation 126b is rendered only on the second interface 125 of the tipping viewer's own viewer device subsystem 110. Other viewers watching the same live stream do not see this particular tipping animation. In this mode, the visual effect data is generated locally by the client application on the viewer device or received from the server 130 and rendered only on the tipping viewer's display.
Mode 2 (Broadcaster-Side Rendering): The tipping animation 126b is rendered on the performer's live broadcast interface on the performer computing device 140, such that the tipping animation is captured as part of the live stream output and is visible to all viewers watching the performer's broadcast. In this mode, the server 130 transmits visual effect instructions (including the direction vector, velocity, image element type, and quantity) to the performer computing device 140, which renders the tipping animation as an overlay on the live broadcast video feed.

In some embodiments, the system supports a third rendering mode (Hybrid Rendering), in which a first visual effect is rendered locally on the tipping viewer's second interface 125 and a second, potentially different, visual effect is rendered on the performer's broadcast interface. For example, the tipping viewer may see a detailed, high-fidelity animation on their own display, while all viewers see a simplified notification animation on the broadcast.

In some embodiments, the rendering mode is selectable by the viewer, by the performer, or by the platform operator through a configuration setting.

### 5.5 Tipping Transaction Binding

As used herein, activating the tipping input includes initiating, confirming, or processing a tipping-related action, including but not limited to generating a tipping event, triggering a transaction, deducting a virtual currency amount, or transmitting a tipping instruction to a server. Each detected gesture event that satisfies the predefined displacement threshold (or each time unit during a press-and-hold gesture) is bound to a tipping transaction processed by the tipping transaction module 133b on the server 130. In a preferred embodiment, each qualifying gesture event corresponds to a deduction of a predefined token amount (e.g., 1 token, 5 tokens, or a viewer-configured amount) from the viewer's account balance. The server 130 validates that the viewer's account balance is sufficient before confirming the transaction. If the balance is insufficient, the server 130 transmits a notification to the viewer device, and the gesture is acknowledged visually on the first interface 120 without completing the tipping transaction. In some embodiments, the activation of the tipping input is conditionally triggered based on the pointing operation satisfying one or more predefined criteria.

In some embodiments, the token amount per gesture event is variable and is determined by one or more kinematic parameters of the gesture. For example, a swipe with a velocity exceeding a first threshold may correspond to 1 token, a swipe with a velocity exceeding a second, higher threshold may correspond to 2 tokens, and so forth. In other embodiments, the token amount is fixed per gesture event, and the total tipping amount is determined by the cumulative number of gesture events performed by the viewer. In some embodiments, the processing of the pointing operation and activation of the tipping input may be performed asynchronously or with a delay, including scenarios in which the pointing operation is recorded and processed at a later time.

### 5.6 Toy Actuation Linkage

When the performer has a connected interactive toy 150, the gesture kinematic parameters extracted from the viewer's swipe gesture are transmitted from the viewer device to the server 130, and the toy command generation module 133d on the server 130 converts the kinematic parameters into toy actuation commands. The toy actuation commands specify at least a vibration intensity level and a vibration duration. In a preferred embodiment, the vibration intensity level is proportional to the swipe velocity V. A faster swipe results in a higher vibration intensity. The vibration duration corresponds to the duration of the swipe gesture or to a predefined pulse duration.

In some embodiments, the toy actuation commands further specify a vibration frequency (e.g., the oscillation frequency of the actuator 153), a vibration pattern (e.g., a single pulse, a double pulse, a ramp-up pattern, or a ramp-down pattern), and a vibration rhythm that corresponds to the temporal pattern of the viewer's successive swipe gestures. For example, if the viewer performs three rapid swipes followed by a pause and then two slow swipes, the toy 150 produces three rapid vibration pulses followed by a pause and then two sustained vibration pulses.

In some embodiments, the mapping between gesture kinematic parameters and toy actuation parameters is defined by a configurable mapping function stored on the server 130. The mapping function may be a linear mapping, a logarithmic mapping, a step function, or a custom curve defined by the platform operator or the performer. In some embodiments, the performer may set minimum and maximum bounds on the vibration intensity and frequency to ensure comfort and safety.

In one aspect, when multiple viewers are simultaneously performing gesture-based tipping directed at the same performer, the server 130 aggregates the toy actuation commands from the multiple viewers. In a preferred embodiment, the aggregation is a weighted sum of the individual actuation commands, capped at a maximum intensity level. In alternative embodiments, the aggregation is a priority queue in which the most recent or highest-intensity command takes precedence, or a time-division multiplexing scheme in which each viewer's commands are applied in sequential time slots.

### 6. Tipping Mode II: Tilt-to-Pour Mode

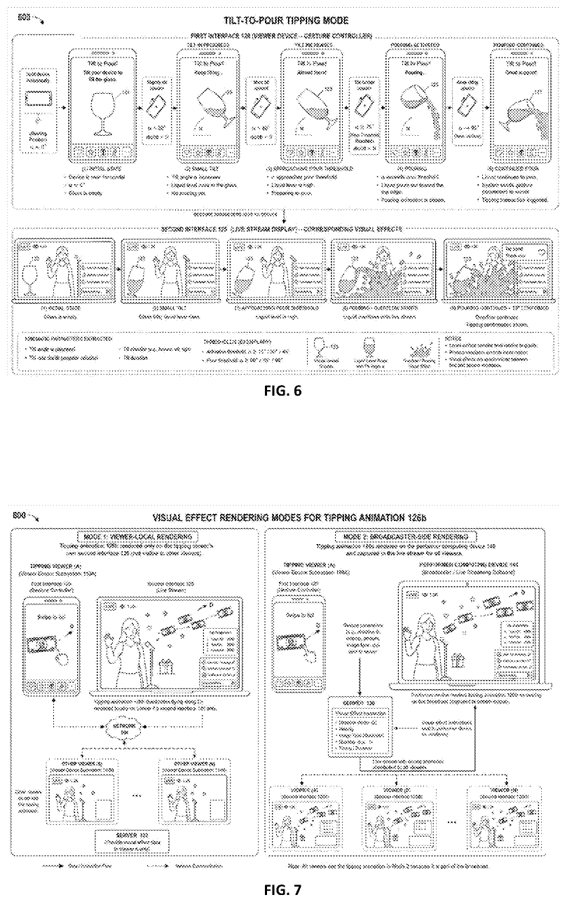

Referring now to FIG. 6, the tilt-to-pour tipping mode is described. In this mode, the viewer holds the viewer device (e.g., a smartphone) in hand and physically tilts the device, simulating the motion of pouring liquid from a vessel.

### 6.1 Gesture Input

The gyroscope 115a and the accelerometer 115b of the viewer device detect the tilt angle α of the device relative to a gravitational reference vector. The gesture detection module monitors the tilt angle α and its rate of change dα/dt. When the tilt angle α exceeds a predefined activation threshold (e.g., 15 degrees, 30 degrees, or 45 degrees from a horizontal resting position), the tilt-to-pour mode is activated. The kinematic parameters extracted include: the tilt angle α, the tilt rate dα/dt (angular velocity), the tilt direction (e.g., forward tilt, lateral tilt), and the tilt duration.

### 6.2 First Interface Visual Feedback

The first interface 120 displays a virtual vessel graphic 123 (e.g., a wine glass, a beer mug, a champagne flute, a cocktail glass, or a generic container). As the viewer tilts the device, the virtual vessel graphic 123 is rendered with a liquid level that rises in response to the tilt angle α. In a preferred embodiment, the liquid level animation is physics-simulated, with the liquid surface remaining approximately level relative to the gravitational vector while the vessel graphic rotates with the device tilt. In some embodiments, the liquid in the virtual vessel graphic 123 has a configurable color and viscosity that affect the animation dynamics (e.g., water flows quickly, honey flows slowly).

When the tilt angle α reaches a pour threshold (e.g., 60 degrees, 75 degrees, or 90 degrees), the first interface 120 renders a pouring animation in which liquid appears to flow out of the virtual vessel graphic 123 toward the edge of the display corresponding to the tilt direction.

### 6.3 Second Interface Visual Feedback

On the second interface 125, the system renders a corresponding visual effect: a large virtual vessel graphic that mirrors the tilt state of the viewer's device. As the viewer tilts, the large virtual vessel on the second interface 125 tilts correspondingly, and the liquid level rises. When the pour threshold is reached, the second interface 125 renders an overflowing liquid animation, with liquid appearing to spill across the live stream display. A tipping notification indicator is displayed to confirm the tipping transaction. In some embodiments, the association between the representation of the tipping input and the host user is preconfigured or dynamically established based on the live streaming session context.

### 6.4 Tipping Transaction Binding

In the tilt-to-pour mode, the tipping transaction is triggered when the tilt angle α exceeds the pour threshold for a continuous duration exceeding a predefined time threshold (e.g., 500 milliseconds, 1 second, or 2 seconds). In some embodiments, the tipping amount is proportional to the duration for which the tilt angle exceeds the pour threshold. In other embodiments, the tipping amount is proportional to the tilt rate dα/dt, such that a faster tilt results in a larger tip. In a further embodiment, the tipping transaction is a continuous stream: for each time unit (e.g., each second) that the tilt angle exceeds the pour threshold, a predefined token amount is deducted from the viewer's account.

### 6.5 Toy Actuation Linkage

The tilt rate dα/dt is mapped to the vibration pulse frequency of the performer-side interactive toy 150. A faster tilt (higher dα/dt) results in a higher vibration pulse frequency. The tilt angle α is mapped to the vibration intensity. A greater tilt angle results in a higher vibration intensity. In some embodiments, the mapping further considers the tilt direction, such that a forward tilt produces a different vibration pattern than a lateral tilt.

### 7. Tipping Mode Iii: Tap-to-burst Mode

In some embodiments, the tap-to-burst tipping mode may also be activated in the system. In this mode, the viewer taps on the image element 121 displayed on the first interface 120.

### 7.1 Gesture Input

The viewer performs a tap gesture (a brief touch-down and touch-up event) on the image element 121. The gesture detection module detects the tap and extracts the following kinematic parameters: the tap position (x, y) on the first interface 120, the tap pressure (where supported), and the tap duration (the time between touch-down and touch-up). In some embodiments, the gesture detection module further detects multi-tap sequences (e.g., double-tap, triple-tap) and extracts the tap frequency (number of taps per unit time).

### 7.2 First Interface Visual Feedback

Upon detection of a tap gesture, the first interface 120 renders a burst animation 124 centered at the tap position. The burst animation 124 depicts the image element 121 exploding or radiating outward from the tap point in a starburst pattern. In a preferred embodiment, the burst animation 124 includes a plurality of smaller image element fragments that fly outward from the tap point and fade out. The intensity of the burst animation (e.g., the number of fragments, the radius of the burst, the speed of the fragments) is proportional to the tap pressure or the tap frequency.

### 7.3 Second Interface Visual Feedback

On the second interface 125, the system renders a corresponding burst visual effect, with image elements radiating outward from a central point on the live stream display. A tipping notification indicator is displayed.

### 7.4 Tipping Transaction Binding

Each tap gesture constitutes one tipping event, bound to a predefined token amount. Rapid successive taps result in rapid successive tipping events.

### 7.5 Toy Actuation Linkage

The tap frequency is mapped to the vibration pulse rate of the toy 150. The tap pressure (where available) is mapped to the vibration intensity per pulse.

### 8. Tipping Mode IV: Flick-to-Launch Mode

In some embodiments, the flick-to-launch tipping mode may also be activated in the system. In this mode, the viewer performs a flick gesture on the image element 121, similar to flicking a coin or a card.

### 8.1 Gesture Input

The viewer places a finger on the image element 121 and performs a rapid, short-distance swipe (a flick). The gesture detection module distinguishes a flick from a full swipe based on the gesture duration and displacement: a flick has a duration below a predefined threshold (e.g., 150 milliseconds) and a displacement below a predefined threshold (e.g., 100 pixels), but a velocity above a predefined threshold (e.g., 500 pixels per second). The kinematic parameters extracted include: the flick direction vector, the flick velocity, and the flick acceleration.

### 8.2 First Interface Visual Feedback

The first interface 120 renders a launch animation in which the image element 121 appears to be flicked off the screen in the flick direction, with a spinning motion and a trajectory arc simulating projectile physics. In some embodiments, the launch animation includes a sound effect synchronized with the flick, such as a coin-flip sound or a card-flick sound.

### 8.3 Second Interface Visual Feedback

On the second interface 125, the system renders a corresponding animation in which the image element appears to land or arrive on the live stream display from the direction corresponding to the flick direction. A tipping notification indicator is displayed.

### 8.4 Tipping Transaction Binding

Each flick gesture constitutes one tipping event. The token amount per flick may be fixed or variable based on the flick velocity.

### 8.5 Toy Actuation Linkage

The flick velocity is mapped to a single vibration pulse intensity on the toy 150. A faster flick produces a sharper, more intense vibration pulse.

### 9. Mode Selection and Combination

In some embodiments, the viewer selects a tipping mode from a mode selection interface rendered on the first interface 120 or on a settings panel accessible from the first interface 120. The mode selection interface presents the available tipping modes (swipe-to-scatter, tilt-to-pour, tap-to-burst, flick-to-launch) as selectable options, each with a descriptive icon and label.

In some embodiments, the viewer may use multiple tipping modes within a single session by switching between modes through the mode selection interface. In other embodiments, the system automatically detects the type of gesture performed by the viewer and activates the corresponding tipping mode without requiring explicit mode selection. For example, if the viewer performs a swipe, the swipe-to-scatter mode is activated; if the viewer tilts the device, the tilt-to-pour mode is activated.

### 10. Method Flow for Gesture-Based Tipping

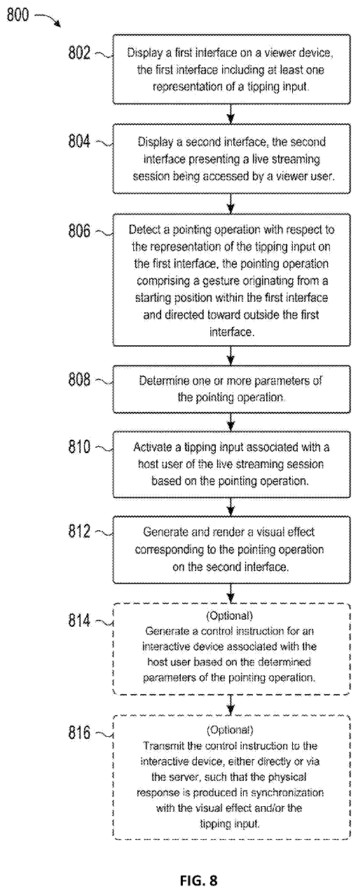

Referring now to FIG. 8, an exemplary method 800 for interactive tipping in a live streaming environment is illustrated. The method 800 may be performed by one or more components of the system 100, including the viewer device subsystem 110 and/or the server 130.

At step 802, a first interface is displayed on a viewer device, the first interface including at least one representation of a tipping input. In some embodiments, the representation of the tipping input comprises an interactive graphical element that is responsive to user input.

At step 804, a second interface is displayed, the second interface presenting a live streaming session being accessed by a viewer user. In some embodiments, the second interface is displayed on the same viewer device as the first interface. In other embodiments, the second interface is displayed on a separate device. The second interface is spatially separate from, and located outside, the first interface.

At step 806, a pointing operation is detected with respect to the representation of the tipping input on the first interface. The pointing operation comprises a gesture originating from a starting position within the first interface and directed toward outside the first interface. In some embodiments, the pointing operation includes a directional input having a spatial vector component.

At step 808, one or more parameters of the pointing operation are determined. The parameters may include kinematic parameters such as direction, velocity, duration, frequency, and/or pressure associated with the pointing operation.

At step 810, a tipping input associated with a host user of the live streaming session is activated based on the pointing operation. In some embodiments, activating the tipping input includes initiating or processing a tipping transaction.

At step 812, a visual effect corresponding to the pointing operation is generated and rendered on the second interface. In some embodiments, the visual effect includes an animation of the representation of the tipping input moving in a direction corresponding to the pointing operation.

At optional step 814, a control instruction is generated for an interactive device associated with the host user based on the determined parameters of the pointing operation. The control instruction causes the interactive device to produce a physical response corresponding to the pointing operation.

At optional step 816, the control instruction is transmitted to the interactive device, either directly or via the server, such that the physical response is produced in synchronization with the visual effect and/or the tipping input.

In some embodiments, one or more of the steps of the method 800 may be performed in a different order, omitted, or combined. In some embodiments, additional steps may be included without departing from the scope of the present disclosure. In some embodiments, if the pointing operation does not satisfy one or more predefined criteria, the method may terminate without activating the tipping input.

In accordance with one aspect of the present disclosure, the interactive tipping mechanism establishes a multi-layer mapping relationship between user input characteristics and system-level semantic interpretation. In particular, the system defines both explicit input dimensions and implicit contextual mappings that collectively determine how a tipping action is interpreted and rendered.

In a first explicit dimension (referred to herein as a first explicit mapping), a spatial direction of a pointing operation performed by the viewer with respect to the representation of the tipping input corresponds to a spatial relationship between the first interface and the second interface. Specifically, the pointing operation originates within the first interface and is directed toward a location outside the first interface, and this directional characteristic is interpreted as indicating a transition from the first interface to the second interface.

In a corresponding implicit dimension (referred to herein as a first implicit mapping), the system interprets the directional component of the pointing operation as representing a transfer or projection of the tipping input from a viewer-side interaction space associated with the first interface to a content consumption space associated with the second interface. In this manner, the relative positioning of the first interface and the second interface defines a semantic spatial framework through which the pointing operation acquires contextual meaning beyond mere gesture input.

In a second explicit dimension (referred to herein as a second explicit mapping), the pointing operation is performed with respect to the representation of the tipping input displayed on the first interface, thereby indicating that the representation of the tipping input is the operative object of the user interaction.

In a corresponding second implicit dimension (referred to herein as a second implicit mapping), the system associates the representation of the tipping input with a host user presented within the second interface, such that activation of the tipping input is directed to the host user as a target entity. In other words, although the pointing operation is physically performed on an object within the first interface, the system resolves the ultimate target of the interaction as an entity represented within the second interface.

Through the combination of the first explicit mapping and the first implicit mapping, the system establishes a directional correspondence between gesture input and cross-interface spatial semantics. Through the combination of the second explicit mapping and the second implicit mapping, the system establishes a functional correspondence between the manipulated graphical element and the recipient of the tipping action.

Accordingly, the interactive tipping mechanism simultaneously encodes (i) a spatial transfer relationship between interfaces and (ii) a target association relationship between user input and a host user, thereby enabling a unified interaction paradigm in which a single pointing operation conveys both directional intent and target attribution across multiple interface contexts.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

It will be apparent to those skilled in the art that various modifications and variations can be made to the disclosed system and method. Other embodiments will be apparent to those skilled in the art from consideration of the specification and practice of the disclosed method and apparatus. It is intended that the specification and examples be considered as exemplary only, with a true scope being indicated by the following claims.

What is claimed is:

1. A system for interactive tipping in a live streaming environment, the system comprising: a memory storing instructions; and a processor coupled to the memory and configured to execute the instructions to:
display, on a first interface of a viewer device, at least one representation of a tipping input;
display, on a second interface, a live streaming session being accessed by a viewer user;
detect, during access to the live streaming session, a pointing operation with respect to the representation of the tipping input on the first interface, wherein the pointing operation comprises a gesture originating from a starting position within the first interface and directed toward outside the first interface, and wherein the second interface is located outside the first interface; and
activate, based on the pointing operation, the tipping input with respect to a host user corresponding to the live streaming session being accessed by the viewer user in the second interface.

2. The system of claim 1, wherein the viewer device comprises a first device and a second device, wherein the first interface is displayed on the first device which is configured to receive the tipping input and the second interface is displayed on the second device which is configured to receive the live streaming session.

3. The system of claim 2, wherein the processor is further configured to:
establish a connection between the first device and the second device to enable the first device to be associated with a tipping function of the live streaming session associated with at least one representation of the tipping input.

4. The system of claim 1, wherein the processor is further configured to generate a tipping effect rendering instruction in response to the detected pointing operation directed to the representation of the tipping input on the first interface, the tipping effect rendering instruction specifying visual characteristics of a tipping effect to be rendered on the second interface.

5. The system of claim 4, wherein the tipping effect comprises an animation of the representation of the tipping input moving from a region corresponding to the viewer user toward a region corresponding to the host user on the second interface.

6. The system of claim 5, wherein a trajectory of the animated representation is determined based on a direction parameter captured from the pointing operation.

7. The system of claim 4, wherein the tipping effect is rendered exclusively on the viewer device and is not visible to other viewer devices accessing the same live streaming session.

8. The system of claim 4, wherein the processor is further configured to transmit the tipping effect rendering instruction to a server associated with the live streaming session, such that the tipping effect is rendered in the live streaming session and is visible to other viewer devices accessing the same live streaming session.

9. The system of claim 1, wherein the processor is further configured to:
generate a control instruction for an interactive device associated with the host user in response to the detected pointing operation directed to the representation of the tipping input on the first interface, the control instruction causing an interactive device associated with the host user to produce a physical response corresponding to kinematic parameters of the pointing operation, the kinematic parameters comprising at least one of force, speed, direction, angle, rhythm, and duration.

10. The system of claim 9, wherein the physical response comprises at least one of vibration intensity, vibration frequency, and vibration pattern, and wherein the physical response is determined based on at least one of the force parameter, the speed parameter, and the rhythm parameter of the pointing operation.

11. The system of claim 9, wherein the control instruction is transmitted to the interactive device in parallel with processing of activating the tipping input.

12. The system of claim 1, wherein the processor is further configured to:
receive a base value for the tipping input; and
determine a tipping amount as an integer multiple of the base value based on kinematic parameters of the pointing operation.

13. The system of claim 1, wherein the representation of the tipping input comprises a currency representation, and the processor is further configured to:
generate a visual feedback on the first interface that dynamically corresponds to the pointing operation;
and wherein the visual feedback comprises an animation of the currency representation moving in a direction corresponding to a direction parameter of the pointing operation.

14. The system of claim 1, wherein the pointing operation either (i) is performed within the first interface and directed toward a boundary of the first interface, or (ii) extends from within the first interface to outside the first interface.

15. The system of claim 1, wherein the pointing operation is directed toward a region corresponding to the second interface.

16. A computer-implemented method for interactive tipping in a live streaming environment, the method comprising:
displaying, on a first interface of a viewer device, at least one representation of a tipping input;
displaying, on a second interface, a live streaming session being accessed by a viewer user;
detecting, during access to the live streaming session, a pointing operation with respect to the representation of the tipping input on the first interface, wherein the pointing operation comprises a gesture originating from a starting position within the first interface and directed toward outside the first interface, and wherein the second interface is located outside the first interface; and
activating, based on the pointing operation, the tipping input with respect to a host user corresponding to the live streaming session being accessed by the viewer user in the second interface.

17. The method of claim 16, wherein the viewer device comprises a first device and a second device, wherein the first interface is displayed on the first device which is configured to receive the tipping input and the second interface is displayed on the second device which is configured to receive the live streaming session, and the method further comprises:
establishing a communication link between the first device and the second device.

18. The method of claim 16, further comprising:
generating a tipping effect rendering instruction in response to the detected pointing operation directed to the representation of the tipping input on the first interface, the tipping effect rendering instruction specifying visual characteristics of a tipping effect to be rendered on the second interface.

19. The method of claim 16, further comprising:
generating a control instruction for an interactive device associated with the host user in response to the detected pointing operation directed to the representation of the tipping input on the first interface, the control instruction causing the interactive device associated with the host user to produce a physical response corresponding to physical parameters of the pointing operation, the physical parameters comprising at least one of force, speed, direction, angle, rhythm, and duration.

20. A non-transitory computer-readable storage medium storing instructions that, when executed by a processor, cause the processor to perform operations comprising:
displaying, on a first interface of a viewer device, at least one representation of a tipping input;
displaying, on a second interface, a live streaming session being accessed by a viewer user;
detecting, during access to the live streaming session, a pointing operation with respect to the representation of the tipping input on the first interface, wherein the pointing operation comprises a gesture originating from a starting position within the first interface and directed toward outside the first interface, and wherein the second interface is located outside the first interface; and
activating, based on the pointing operation, the tipping input with respect to a host user corresponding to the live streaming session being accessed by the viewer user in the second interface.
